(19) United States
(12) Patent Application Publication
(10) Pub. No.: US 2011/0098748 A1
(43) Pub. Date: Apr. 28, 2011

(54) ADJUSTABLE VERTEBRAL ROD SYSTEM AND METHODS OF USE

(75) Inventor: Jayant Jangra, Memphis, TN (US)

(73) Assignee: Warsaw Orthopedic, Inc., Warsaw, IN (US)

(21) Appl. No.: 12/605,392

(22) Filed: Oct. 26, 2009

(51) Int. Cl. A61B 17/70

(52) U.S. Cl. 606/278

(57) ABSTRACT

A vertebral rod includes a first elongated section having a first end and a second end. The second end includes an attachment part. A second elongated section has a first end and a second end. The second end includes an attachment part. A connecting plate is disposed for attaching the second end of the first section with the second end of the second section. The connecting plate has a first surface configured for mating engagement with the attachment part of the first section and a second surface configured for mating engagement with the attachment part of the second section.

### TECHNICAL FIELD

The present disclosure generally relates to medical devices for the treatment of spinal disorders, and more particularly to a vertebral rod system including a connecting plate in a configuration such that the vertebral rod system is adjustable for employment in various applications including load sharing, motion preserving and/or rigid fixation applications.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders include discectomy, laminectomy, fusion and implantable prosthetics. As part of these surgical treatments, connecting elements such as vertebral rods are often used to provide stability to a treated region. During surgical treatment, one or more rods may be attached to the exterior of two or more vertebral members.

Rods redirect stresses away from a damaged or defective region while healing takes place to restore proper alignment and generally support the vertebral members. In some applications, rods are attached to the vertebral members without the use of implants or spinal fusion. The disclosure describes an improvement over these prior art technologies.

### SUMMARY OF THE INVENTION

Accordingly, a vertebral rod system is provided including a connecting plate in a configuration such that the vertebral rod system is adjustable for employment in various applications including load sharing, motion preserving and/or rigid fixation applications.

In one particular embodiment, in accordance with the principles of the present disclosure, a vertebral rod is provided. The vertebral rod includes a first elongated section having a first end and a second end. The second end includes an attachment part. A second elongated section has a first end and a second end. The second end includes an attachment part. A connecting plate is disposed for attaching the second end of the first section with the second end of the second section. The connecting plate has a first surface configured for mating engagement with the attachment part of the first section and a second surface configured for mating engagement with the attachment part of the second section.

In one embodiment, the second end of the first section includes a cylindrical head defining a transverse surface including the attachment part. The second end of the second section includes a cylindrical head defining a transverse surface including the attachment part of the second section.

The attachment part of the first section can have a splined configuration. The attachment part may include a plurality of teeth disposed radially about the transverse surface. The attachment part of the second section may have a splined configuration.

In one embodiment, the connecting plate includes a cylindrical washer. The first surface and the second surface of the connecting plate can have a splined configuration. The first surface and the second surface of the connecting plate may have a plurality of teeth disposed radially thereabout. The connecting plate can be fabricated from a material having a low durometer. The connecting plate can also be fabricated from a material having a high durometer.

The first section and the second section may be relatively rotatable through an angle of 360°. The first section and the second section may also be selectively fixable within the angle of 360° via a locking part.

In one embodiment, the vertebral rod includes a first rod extending to a cylindrical head that defines a transverse surface. The transverse surface includes a plurality of gear teeth disposed radially thereabout. A second rod extends to a cylindrical head that defines a transverse surface. The transverse surface includes a plurality of gear teeth disposed radially thereabout. A connecting washer is disposed to attach the head of the first rod with the head of the second rod. The connecting washer includes a first surface having a splined configuration and a second opposing surface having a splined configuration. The splined configuration of the first and second opposing surfaces matingly engage the gear teeth of the first and second rods to attach the first rod with the second rod.

In one embodiment, an adjustable vertebral rod system is provided. The adjustable vertebral rod system includes a vertebral rod as discussed, and a plurality of connecting plates. Each of the connecting plates are separately and independently configured for attaching the second end of the first section with the second end of the second section. Each connecting plate has a first surface configured for mating engagement with the attachment part of the first section and a second surface configured for mating engagement with the attachment part of the second section.

The plurality of connecting plates includes at least a first plate having a low durometer for a motion preservation application, a second plate having an intermediate durometer for a load sharing application, and a third plate including a metal material for a rigid fixation application. Additional connecting plates are also contemplated.

### BRIEF DESCRIPTION OF THE DRAWINGS

The present disclosure will become more readily apparent from the specific description accompanied by the following drawings, in which:

FIG. 1 is a perspective view of one embodiment of a vertebral rod in accordance with the principles of the present disclosure;

FIG. 2 is a perspective cutaway view of the vertebral rod shown in FIG. 1 with parts separated;

FIG. 3 is an enlarged perspective view of a connecting plate of the vertebral rod shown in FIG. 1;

FIG. 4 is a side cross-section cutaway view of the vertebral rod shown in FIG. 1; and

FIG. 5 is a side, cross section view of the vertebral rod shown in FIG. 1 fastened to vertebrae.

Like reference numerals indicate similar parts throughout the figures.

### DETAILED DESCRIPTION OF THE INVENTION

The exemplary embodiments of the vertebral rod system and methods of use disclosed are discussed in terms of medical devices for the treatment of spinal disorders and more particularly, in terms of a vertebral rod system including a connecting plate in a configuration such that the vertebral rod system is adjustable for employment in various applications including load sharing, motion preserving and/or rigid fixation applications. It is envisioned that the vertebral rod system and methods of use disclosed provide stability and maintains structural integrity while reducing stress on spinal elements. It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is further envisioned that the present disclosure may be employed with surgical treatments including open surgery and minimally invasive procedures of such disorders, such as, for example, discectomy, laminectomy, fusion, bone graft and implantable prosthetics. It is contemplated that the present disclosure may be employed with other osteal and bone related applications, including those associated with diagnostics and therapeutics. It is further contemplated that the disclosed vertebral rod system may be employed in a surgical treatment with a patient in a prone or supine position, employing a posterior, lateral or anterior approach. The present disclosure may be employed with procedures for treating the lumbar, cervical, thoracic and pelvic regions of a spinal column. It is envisioned that the present disclosure may be employed for load sharing and motion preservation via adjustability of a vertebral rod for posterior spinal stabilization.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left, and right, are for illustrative purposes only and can be varied within the scope of the disclosure. For example, the references “upper” and “lower” are relative and used only in the context to the other, and are not necessarily “superior” and “inferior”.

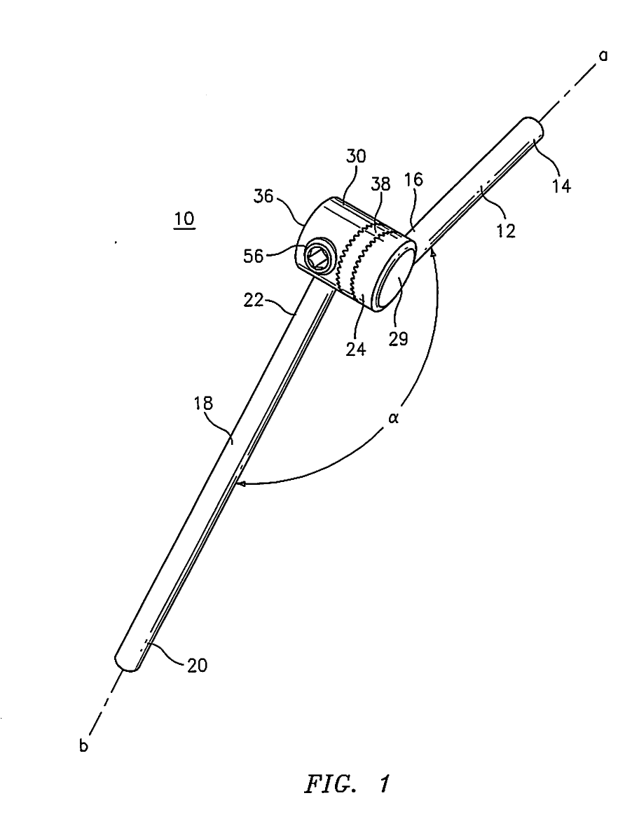

The following discussion includes a description of a vertebral rod system, related components and exemplary methods of employing the vertebral rod system in accordance with the principles of the present disclosure. Alternate embodiments are also disclosed. Reference will now be made in detail to the exemplary embodiments of the present disclosure, which are illustrated in the accompanying figures. Turning now to FIGS. 1-5, there are illustrated components of a vertebral rod system in accordance with the principles of the present disclosure.

The components of the vertebral rod system are fabricated from materials suitable for medical applications, including metals, polymers, ceramics, biocompatible materials and/or their composites, depending on the particular application and/or preference of a medical practitioner. For example, a vertebral rod, discussed below, of the vertebral rod system can be fabricated from materials such as commercially pure titanium, titanium alloys, super-elastic titanium alloys, cobalt-chrome alloys, stainless steel alloys, thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ polymeric rubbers composites, biocompatible materials such as polymers including plastics, metals, ceramics and composites thereof, rigid polymers including polyphenylene, polyamide, polyimide, polyetherimide, polyethylene, polyurethane, epoxy and silicone. Different sections of the rod may have alternative material composites to achieve various desired characteristics such as strength, rigidity, elasticity, compliance, biomechanical performance, durability and radiolucency or imaging preference.

As a further example, a connecting plate of the vertebral rod system may be fabricated from materials such as silicone, polyurethane, silicone-polyurethane copolymers, polymeric rubbers, polyolefin rubbers, hydrogels, semi-rigid and rigid materials, and biocompatible materials such as elastomers, rubbers, thermoplastic elastomers, thermoset elastomers, elastomeric composites and plastics. It is envisioned that the connecting plate can also be manufactured from, for example, titanium (Ti) alloys, Cobalt-Chrome-Molybdenum (Co-Ch-Mo) alloys, Grade 5 titanium (Ti-6Al-4V), Commercially Pure Titanium (CP Ti), cobalt-chromium (Co-Cr), stainless steel, Nitinol, and/or carbon-reinforced PEEK to provide, for example, a rigid connection. One skilled in the art, however, will realize that such materials and fabrication methods suitable for assembly and manufacture, in accordance with the present disclosure, would be appropriate.

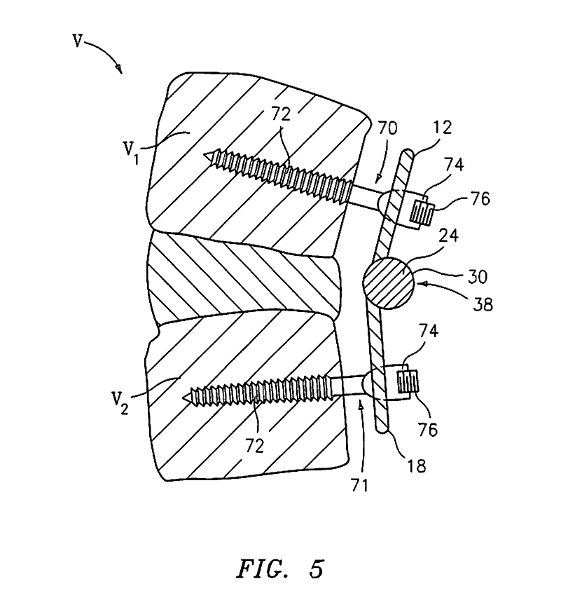

The vertebral rod system is configured for attachment to vertebrae (as shown, for example, in FIG. 5) during surgical treatment of a spinal disorder, examples of which are discussed herein. The vertebral rod system has a vertebral construct 10, which includes a first elongated section, such as, for example, upper rod 12 that defines a longitudinal axis a, and extends from a first end 14 to a second end 16. A second elongated section, such as, for example, lower rod 18 defines a longitudinal axis b, and extends from a first end 20 to a second end 22. It is contemplated that the respective cross-section of rods 12, 18 may have various configurations, for example, round, oval, rectangular, polygonal, irregular, uniform and non-uniform. Rod 12 may have a different cross-sectional area, geometry, material or material property such as strength, modulus or flexibility relative to rod 18.

Second end 16 includes a head 24 having a cylindrical configuration. Head 24 has an attachment part, as will be discussed, including a surface 26, which is oriented transverse to axis a. Surface 26 has a plurality of gear teeth 28 disposed radially thereabout in a splined configuration. Head 24 includes an outer surface 29. It is envisioned that head 24 may be monolithically formed, integrally connected or arranged with attaching elements for connection with rod 12.

Second end 22 includes a head 30 having a cylindrical configuration. Head 30 has an attachment part including a surface 32, which is oriented transverse to axis b. Surface 32 has a plurality of gear teeth 34 disposed radially thereabout in a splined configuration. Head 30 includes an outer surface 36. It is envisioned that head 24 may be monolithically formed, integrally connected or arranged with attaching elements for connection with rod 18. It is envisioned that head 24 and/or head 30 may have one of a plurality of surfaces having a splined connector configuration.

It is contemplated that the respective cross-section of heads 24, 30 may have various configurations, for example, round, oval, rectangular, polygonal, irregular, uniform and non-uniform. Head 24 may have a different cross-sectional area, geometry, material or material property such as strength, modulus or flexibility relative to head 30.

A connecting plate, such as, for example, a connecting washer 38 is connected with rods 12, 18 and disposed therebetween as a joining section of the components of vertebral construct 10. Connecting washer 38 is disposed for connecting head 24 to head 30. It is envisioned that the components of vertebral construct 10 may be monolithically formed, integrally connected or arranged with attaching elements.

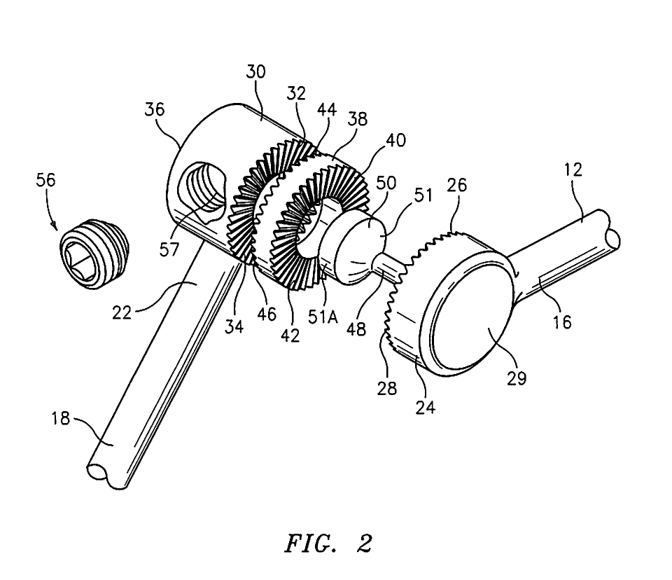

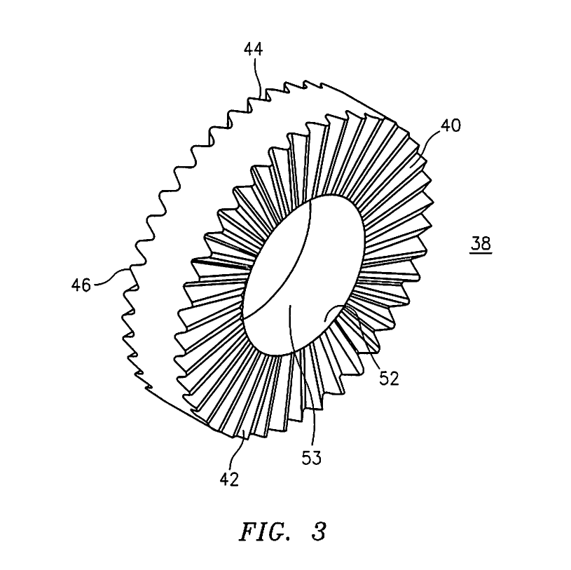

Connecting washer 38 includes a first surface 40 having a splined configuration with a plurality of gear teeth 42 disposed radially thereabout and a second opposing surface 44 having a splined configuration with a plurality of gear teeth 46 disposed radially thereabout, as shown in FIG. 3. The splined configuration of surface 40 including gear teeth 42 is configured to matingly engage gear teeth 28, and surface 44 including gear teeth 46 is configured to matingly engage gear teeth 34 respectively to attach rods 12, 18, as shown in FIG. 2. Connecting washer 38 may be fabricated from a material of high durometer or low durometer to provide a range of stiffness to connecting washer 38. In high durometer applications, vertebral construct 10 may be configured as a load sharing device, and for low durometer applications, vertebral construct 10 may be configured for flexibility to allow motion. Vertebral construct 10 may be employed for a particular stiffness and/or flexibility during modes of movement including flexion, extension, axial rotation and/or lateral bending for load sharing, motion preservation and/or rigid fixation applications.

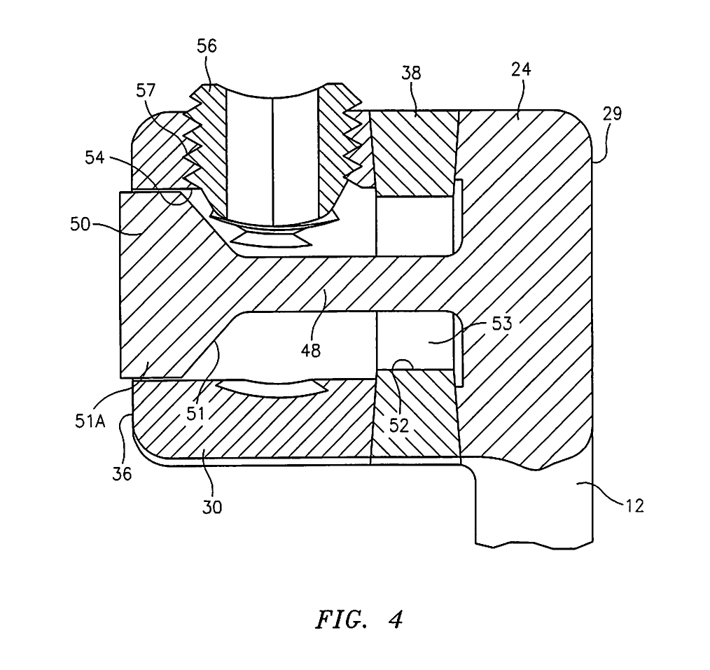

In assembly of the components of vertebral construct 10, head 24 includes an elongated shaft 48 that extends to a retainer 50, as shown in FIGS. 2 and 4. Shaft 48 has a reduced diameter or thickness and extends to retainer 50, which includes a gradual or tapering increase in diameter or thickness such as frustro conical surface 51. Surface 51 extends to an enlarged portion 51A of retainer 50, which is configured for disposal within head 30, as will be discussed. Connecting washer 38 includes an inner surface 52 defining a cavity 53 for disposal of shaft 48 therein and extension therethrough.

Head 30 includes a bearing cavity 54, which is configured for disposal of shaft 48 and slidable support of retainer 50 for assembly such that the gear teeth of connecting washer 38 mate with the gear teeth of rods 12, 18. It is contemplated that all or any portions of shaft 48 and/or retainer 50 may be solid, hollow, porous or combinations thereof. Shaft 48 and retainer 50 may be monolithically formed, integrally connected or arranged with attaching elements. Alternatively, shaft 48 may have an enlarged diameter or thickness in all or sections thereof, and retainer 50 may have alternate configurations such as a block, cylinder drum or spaced apart sections. Shaft 48 and retainer 50 have a cylindrical cross section, however, shaft 48 and retainer 50 may have alternative configurations such as polygonal, rectangular and oval.

Head 30 includes a locking part such as a set screw 56 that is received within a cavity 57. Cavity 57 is threaded to receive set screw 56. For assembly of the components of vertebral construct 10, head 24, a selected connecting washer 38 and head 30 are assembled such that shaft 48 is disposed within cavity 53 and retainer 50 is slidably disposed and engaged with bearing cavity 54. Upon placement of these components, set screw 56 is threaded with cavity 57 and drawn into bearing cavity 54. Set screw 56 is drawn to engage surface 51 such that the components of vertebral construct 10 are drawn together and secured as a unit. Set screw 56 may be threadedly removed from head 30 to disassemble the components of vertebral construct 10 to, for example, alternate a selected connecting washer 38 for use, as will be discussed. Assembly of the components can be performed prior to delivery to a surgical site and/or in situ, including subsequent to fixation of rods 12, 18 with vertebrae.

The mating configuration of connecting washer 38 facilitates assembly of the components of adjustable vertebral construct 10 such that the device may be employed in a plurality of applications including, for example, load sharing, motion preserving and/or rigid fixation through use of an implant kit or vertebral rod system including a plurality of connecting washers, as will be discussed below.

It is envisioned that connecting washer 38 may have a wide, narrow, round or irregular configuration. Connecting washer 38 may be formed of one or a plurality of elements such as spaced apart and/or staggered portions. Connecting washer 38 may be fabricated from the same or alternative material to rods 12, 18. Connecting washer 38 may also have a different cross-sectional area, geometry or material property such as strength, modulus and flexibility relative to rods 12, 18.

In one embodiment, only one of opposing surfaces 40, 44 of connecting washer 38 may be configured to matingly engage with the attachment part of either rods 12, 18. Connecting washer 38 may be additionally or alternatively connected to rods 12, 18 using various methods and structure including molding of a continuous component, mechanical fastening, adhesive bonding and combinations thereof. It is further envisioned that particular parameters of rods 12, 18, and connecting washer 38 may be selected to modulate the flexibility or stiffness of the vertebral rod system including the cross-sectional area (or thickness) of connecting washer 38, material modulus of rods 12, 18 and connecting washer 38, as well as rod material properties. These parameters allow modification of the properties or performance of the vertebral rod system such as strength, durability, flexibility (or stiffness), overall profile and the ability to employ a percutaneous approach, for a particular application. Connecting washer 38 may have a variable thickness, according to the requirements of the particular application. It is envisioned that the thickness of connecting washer 38 may be varied to achieve a particular stiffness or flexibility of vertebral construct 10.

It is contemplated that connecting washer 38, and rods 12, 18 may be oriented in alignment, non-aligned, offset, facing or not facing vertebrae and alternate angular orientation.

The adjustability of vertebral construct 10 also includes relative rotation of rods 12, 18 through an angle of 360 degrees. During assembly, as discussed, rod 12 (axis a) can be rotated to a selected angle α relative to rod 18 (axis b). Head 24 is rotated relative to head 30 such that the elongated portion of rod 12 can be rotated clockwise and counter-clockwise. Head 30 is rotated such that the elongated portion of rod 18 can be rotated clockwise and counter-clockwise. As such, axis a of rod 12 can be oriented selectively at angle α in a range from 0 to 360 degrees relative to axis b of rod 18. It is contemplated that, depending on the relative flexibility of surfaces 40, 44 of connecting washer 38 and the attachment parts of rods 12, 18, rods 12 and rod 18 may be rotated relative to the other rod prior to delivery to a surgical site and/or in situ, including subsequent to fixation of rods 12, 18 with vertebrae.

During rotation of rods 12, 18, rod 12 and rod 18 are selectively fixable with the angle of 360 degrees. Axis a of rod 12 is disposed at angle α in a range of 0 to 360 degrees relative to axis b of rod 18, and selectively fixable via set screw 56. Set screw 56 is threaded with head 30 to engage surface 51, as discussed, to secure the selected angular orientation a of rod 12 relative to rod 18.

In one embodiment, an adjustable vertebral rod system includes a kit or set that includes vertebral construct 10 described above and a plurality of alternate connecting washers 38. Each of connecting washers 38 is separately and independently configured for connecting head 24 with head 30, as described above. The plurality of connecting washers 38 include a first washer having a low durometer for a motion preservation application. The plurality of connecting washers 38 also includes a second washer having an intermediate durometer for a load sharing application. The plurality of connecting washers 38 also includes a third washer having a high durometer material, such as a metal material, for a rigid fixation application. These alternate washers 38 may be delivered to an implanted construct 10 such that the washer can be changed out, or implanted with a pre-assembled construct 10, or can be assembled with a construct 10 in situ. It is envisioned that the implant set or kit may include different combinations of the connecting washers disclosed, including sets of one, two, three or other pluralities of alternate and/or similarly configured washers.

The implant set or kit of the vertebral rod system may be used as a load sharing device, a motion preserving device and/or a rigid fixation device. This adjustable configuration, at least in part, is facilitated by changing connecting washer 38. The implant set or kit could include multiple connecting washers 38 of alternate thickness, alternate stiffness and alternate materials allowing for ease of selection with low inventory. The implant set or kit may also include a plurality of connecting washers 38 having the same stiffness, material composition and/or material/mechanical properties. It is envisioned that adjusting the thickness and/or material/mechanical properties of connecting washer 38, the stiffness and/or flexibility of vertebral construct 10 can be altered in the modes of movement of vertebral construct 10 including flexion, extension, axial rotation and/or lateral bending, for load sharing, motion preservation and/or rigid fixation applications. It is further envisioned that load sharing, motion preservation or rigid posterior fixation can be obtained from the components of the vertebral rod system as a stand alone kit or set. It is contemplated that the vertebral rod system includes relative positioning and angulation of the rods, which can be integrated with dynamic stabilization.

In assembly, operation and use, the adjustable vertebral rod system is employed with a surgical procedure for treatment of a spinal disorder affecting a section of a spine of a patient, as discussed herein. The adjustable vertebral rod system may also be employed with other surgical procedures. In particular, the adjustable vertebral rod system is employed with a surgical procedure for treatment of a condition or injury of an affected section of the spine including vertebrae V, as shown in FIG. 5. It is contemplated that the vertebral rod system is attached to vertebrae V for stabilization of the affected section of the spine to facilitate healing and therapeutic treatment.

In use, to treat the affected section of the spine, a medical practitioner obtains access to a surgical site including vertebra V in any appropriate manner, such as through incision and retraction of tissues. It is envisioned that the adjustable vertebral rod system may be used in any existing surgical method or technique including open surgery, mini-open surgery, minimally invasive surgery and percutaneous surgical implantation, whereby the vertebrae V is accessed through a mini-incision, or a sleeve that provides a protected passageway to the area. Once access to the surgical site is obtained, the particular surgical procedure is performed for treating the spinal disorder. The adjustable vertebral rod system is then employed to augment the surgical treatment. The adjustable vertebral rod system can be delivered or implanted as a pre-assembled device or can be assembled in situ.

A first fastening element, such as, for example, fixation screw assembly 70 is configured to attach upper rod 12 to vertebra V₁. A second fastening element, such as, for example, fixation screw assembly 71 is configured to attach lower rod 18 to adjacent vertebra V₂. Pilot holes are made in vertebrae V₁, V₂ for receiving fixation screw assemblies 70, 71. Fixation screw assemblies 70, 71 include threaded bone engaging portions 72 that are inserted or otherwise connected to vertebrae V₁, V₂, according to the particular requirements of the surgical treatment. Fixation screw assemblies 70, 71 each have a head 74 with a bore, or through opening and a set screw 76, which is torqued on to rods 12, 18 to attach construct 10 in place with vertebrae V.

As described, head 30 slidably receives retainer 50 and set screw 56 engages surface 51 to assemble vertebral construct 10 as a unit of the adjustable vertebral rod system. As set screw 56 is threaded and drawn into locking engagement with retainer 50, gear teeth 42 of connecting washer 38 matingly engage with gear teeth 28 of head 24. Gear teeth 34 of head 30 matingly engage with gear teeth 46 of connecting washer 38 for assembly of rods 12, 18. The mating configuration of connecting washer 38 facilitates assembly of the components of adjustable vertebral construct 10 such that the device may be employed in a plurality of applications including, for example, load sharing, motion preserving and/or rigid fixation through use of the vertebral rod system including the plurality of connecting washers 38 described above.

The orientation of rods 12, 18 can be adjusted and selectively fixable with the angle of 360 degrees. This facilitates adjustment of vertebral construct 10 to account for varying anatomical geometry and angles, for example, of lordosis and/or kyphosis depending upon the anatomical region and/or pathological considerations. This avoids the need for bending of the vertebral construct to accommodate such anatomical considerations. This also avoids comprising the strength of a vertebral rod construct that may occur during bending of a vertebral construct and/or avoid surface notches that would be inflicted to a vertebral construct by bending instruments. Rod 12 along axis a is disposed at angle α, which may, for example, be in a range of 20 to 50 degrees for a two level lumbar construct, relative to rod 18 along axis b and selectively fixable via set screw 56. Angle α can be varied depending on patient anatomy, pathology and/or the surgical procedure employed.

The vertebral rod system can be used with various bone screws, pedicle screws or multi-axial screws used in spinal surgery. It is contemplated that the vertebral rod system may be used with pedicle screws coated with an osteoconductive material such as hydroxyapatite and/or osteoinductive agent such as a bone morphogenic protein for enhanced bony fixation to facilitate motion of the treated spinal area. The components of the vertebral rod system can be made of radiolucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used, such as being disposed at the end portions of vertebral construct 10.

It is contemplated that rods 12, 18 have an arcuate configuration and an increased length providing the ability to extend over two or more intervertebral levels. It is contemplated that the configuration of the vertebral rod system may provide dynamic or flexible stabilization over a plurality of intervertebral levels, including treated and untreated vertebral and intervertebral levels. It is further contemplated that lower rod 18 provides a less flexible, or more rigid stabilization relative to upper rod 12. It is envisioned that lower rod 18 may be attached with vertebrae across lower lumbar levels such as levels L5-S1. Lower rod 18 may be cut or trimmed during a surgical procedure such that the size of vertebral construct 10 can be modified according to patient needs or the particular requirements of a surgical treatment or medical practitioner.

It is envisioned that the vertebral construct 10 may be heat treated during surgery to obtain a best fit curvature or shape for the patient. It is further envisioned that vertebral construct 10 may include one or a plurality of head 24, 30/washer 38 sections spaced along the length of construct 10.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A vertebral rod comprising:
a first elongated section having a first end and a second end, the second end including an attachment part;
a second elongated section having a first end and a second end, the second end including an attachment part; and
a connecting plate disposed for attaching the second end of the first section with the second end of the second section, the connecting plate having a first surface configured for mating engagement with the attachment part of the first section and a second surface configured for mating engagement with the attachment part of the second section.

2. A vertebral rod according to claim 1, wherein the attachment part of the first section has a splined configuration.

3. A vertebral rod according to claim 2, wherein the attachment part of the second section has a splined configuration.

4. A vertebral rod according to claim 1, wherein the second end of the first section includes a cylindrical head defining a transverse surface including the attachment part.

5. A vertebral rod according to claim 4, wherein the attachment part includes a plurality of teeth disposed radially about the transverse surface.

6. A vertebral rod according to claim 4, wherein the second end of the second section includes a cylindrical head defining a transverse surface including the attachment part of the second section.

7. A vertebral rod according to claim 5, wherein the attachment part includes a plurality of teeth disposed radially about the transverse surface of the second section.

8. A vertebral rod according to claim 1, wherein the connecting plate includes a cylindrical washer.

9. A vertebral rod according to claim 1, wherein the first surface and the second surface of the connecting plate have a splined configuration.

10. A vertebral rod according to claim 1, wherein the first surface and the second surface of the connecting plate have a plurality of teeth disposed radially thereabout.

11. A vertebral rod according to claim 1, wherein the connecting plate is fabricated from a material having a low durometer.

12. A vertebral rod according to claim 1, wherein the connecting plate is fabricated from a material having a high durometer.

13. A vertebral rod according to claim 1, wherein the first section and the second section are relatively rotatable through an angle of 360°.

14. A vertebral rod according to claim 13, wherein the first section and the second section are selectively fixable within the angle of 360° via a locking part.

15. A vertebral rod comprising:
a first rod extending to a cylindrical head that defines a first transverse surface, the first transverse surface including a plurality of gear teeth disposed radially thereabout;
a second rod extending to a cylindrical head that defines a second transverse surface, the second transverse surface including a plurality of gear teeth disposed radially thereabout; and
a connecting washer disposed to attach the head of the first rod with the head of the second rod, the connecting washer including a first surface having a splined configuration and a second opposing surface having a splined configuration, wherein the splined configuration of the first and second opposing surfaces matingly engage the gear teeth of the first and second rods to attach the first rod with the second rod.

16. A vertebral rod according to claim 15, wherein the first rod is rotatable through an angle of 360° relative to the second rod.

17. A vertebral rod according to claim 16, wherein the first rod is selectively fixable within the angle of 360° relative to the second rod.

18. An adjustable vertebral rod system comprising:
a first elongated section having a first end and a second end, the second end including an attachment part;
a second elongated section having a first end and a second end, the second end including an attachment part; and
a plurality of connecting plates, each of the connecting plates being separately and independently configured for attaching the second end of the first section with the second end of the second section, each connecting plate having a first surface configured for mating engagement with the attachment part of the first section and a second surface configured for mating engagement with the attachment part of the second section;
the plurality of connecting plates including:
a first plate having a low durometer for a motion preservation application,
a second plate having an intermediate durometer for a load sharing application, and
a third plate including a metal material for a rigid fixation application.

19. A vertebral rod according to claim 18, wherein the first section and the second section are relatively rotatable through an angle of 360°.

20. A vertebral rod according to claim 18, wherein the first section and the second section are selectively fixable within the angle of 360° via a locking part.
